(19) United States
(12) Patent Application Publication
(10) Pub. No.: US 2024/0342104 A1
(43) Pub. Date: Oct. 17, 2024

(54) DOSAGE FORM FOR IMPROVING PALATABILITY OF DRUG SUBSTANCE

(71) Applicant: ACER THERAPEUTICS, INC., Newton, MA (US)

(72) Inventors: Leah E. Appel, Bend, OR (US); Joshua R. Shockey, Bend, OR (US); D. Christopher Schelling, Bend, OR (US)

(21) Appl. No.: 18/682,481

(22) PCT Filed: Aug. 11, 2022
(86) PCT No.: PCT/US2022/040082

§ 371 Date: Feb. 9, 2024

Related U.S. Application Data
(60) Provisional application No. 63/232,011, filed on Aug. 11, 2021.

(51) Int. Cl. A61K 9/50

(52) U.S. Cl. CPC A61K 9/5078

(57) ABSTRACT

Provided is a dosage form, comprising a core geometric entity, a drug geometric entity connected with the core geometric entity, and a taste-masking geometric entity surrounding the drug geometric entity. The drug geometric entity comprises a drug substance, and the taste-masking geometric entity inhibits the rapid release of the drug substance from the dosage form when the dosage form is administered orally to the subject.

### FIELD OF THE INVENTION

The present disclosure generally relates to a dosage form for improving palatability of a drug substance.

### BACKGROUND

Many drug substances are unpalatable due to the highly unpleasant taste, which may lead to non-compliance, insufficient dosing and suboptimal outcomes. For example, sodium 4-phenylbutyrate (often referred to simply as “sodium phenylbutyrate” or NaPB) is a drug used primarily for the treatment of urea cycle disorders. The sodium phenylbutyrate is administered orally, and the effective treatment requires a relatively large dosage, typically 5-20 grams per day, often for the patient's entire life. Unfortunately, sodium phenylbutyrate has an extremely unpleasant taste and is highly unpalatable, and consequently, patient compliance is often low.

The present invention addresses the need to improve patient compliance, by providing a dosage form for improving palatability of the drug substance.

### SUMMARY

The present disclosure in one aspect provides a dosage form for improving palatability of a drug substance. The dosage form includes multiple entities connected together to inhibit the rapid release of the drug substance and allow the release of the drug substance in the stomach of the subject, so as to mask the unpleasant taste of the drug substance.

In some aspects, the dosage form comprises a core geometric entity, a drug geometric entity connected with the core geometric entity, and a taste-masking geometric entity surrounding the drug geometric entity. The drug geometric entity comprises a drug substance, and the taste-masking geometric entity inhibits the release of the drug substance from the dosage form in the mouth of a subject when the dosage form is administered orally to the subject.

In some aspects, the dosage form has a shape selected from the group consisting of a sphere shape, a tablet shape, a capsule shape, a rod shape, a cubic or cuboidal shape, a pie shape, a cone shape, a pyramid shape, a cylindrical shape, a triangular or polygonal prism shape, and a tetrahedron shape.

In some aspects, the dosage form has a volume of about 3.7×10⁷ μm³ to 1.25×10⁸ μm³. In some aspects, the dosage form has a volume of about 7×10⁷ μm³.

In some aspects, the dosage form has a diameter or maximum length of about 334 μm to 500 μm. In some aspects, the dosage form has a diameter or maximum length of about 417 μm.

In some aspects, the core geometric entity has a shape selected from the group consisting of a sphere shape, a tablet shape, a capsule shape, a rod shape, a cubic or cuboidal shape, a pie shape, a cone shape, a pyramid shape, a cylindrical shape, a triangular or polygonal prism shape, and a tetrahedron shape.

In some aspects, the core geometric entity has a volume of about 4.6×10⁶ m³ to 1.5×10⁷ μm³. In some aspects, the core geometric entity has a volume of about 9×10⁶ m³.

In some aspects, the core geometric entity has a diameter or maximum length of about 166 μm to 248 μm. In some aspects, the core geometric entity has a diameter or maximum length of about 207 μm.

In some aspects, the drug geometric entity surrounds the core geometric entity.

In some aspects, the drug geometric entity has an average thickness of about 77 μm to 115 μm. In some aspects, the drug geometric entity has an average thickness of about 96 μm.

In some aspects, the taste-masking geometric entity has an average thickness of about 7 μm to 11 μm. In some aspects, the taste-masking geometric entity has an average thickness of about 9 μm.

In some aspects, the dosage form further comprises a first sealing geometric entity applied between the drug geometric entity and the taste-masking geometric entity, wherein the first sealing geometric entity prevents or reduces direct contact of the drug geometric entity and the taste-masking geometric entity. In some aspects, the first sealing geometric entity has an average thickness of about 1 μm.

In some aspects, the dosage form further comprises a second sealing geometric entity applied between the core geometric entity and the drug geometric entity, wherein the second sealing geometric entity prevents or reduces direct contact of the core geometric entity and the drug geometric entity. In some aspects, the second sealing geometric entity has an average thickness of about 1 μm.

In some aspects, the dosage form further comprises a coating geometric entity surrounding the taste-masking geometric entity, wherein the coating geometric entity prevents or reduces deterioration of the dosage form by moisture in the air.

In other aspects, the dosage form comprises a core geometric entity, a drug geometric entity surrounding the core geometric entity, a first sealing geometric entity surrounding the drug geometric entity, and a taste-masking geometric entity surrounding the first sealing geometric entity. The drug geometric entity comprises a drug substance, the first sealing geometric entity prevents or reduces direct contact of the drug geometric entity and the taste-masking geometric entity, and the taste-masking geometric entity inhibits the release of the drug substance from the dosage form in the mouth of a subject when the dosage form is administered orally into the subject.

In other aspects, the present disclosure provides a plurality of dosage forms each having a structure as disclosed herein. In some aspects, each dosage form comprises a core geometric entity, a drug geometric entity connected with the core geometric entity, and a taste-masking geometric entity surrounding the drug geometric entity. The drug geometric entity included in each dosage form comprise a drug substance, and the taste-masking geometric entity included in each dosage form inhibits the rapid release of the drug substance from the dosage forms when the dosage form is administered orally to the subject.

### BRIEF DESCRIPTION OF DRAWINGS

The accompanying drawings, which are incorporated herein, form part of the specification. Together with this written description, the drawings further serve to explain the principles of, and to enable a person skilled in the relevant art(s), to make and use the present invention.

FIG. 1 illustrates an exemplary dosage form according to an embodiment of the present invention.

FIG. 2 illustrates an exemplary dosage form according to another embodiment of the present invention.

FIG. 3 illustrates an exemplary dosage form according to another embodiment of the present invention.

FIG. 4 illustrates an exemplary dosage form according to another embodiment of the present invention.

FIG. 5 illustrates an exemplary dosage form according to another embodiment of the present invention.

FIG. 6 illustrates an exemplary dosage form according to another embodiment of the present invention.

FIG. 7 illustrates an exemplary dosage form according to another embodiment of the present invention.

### DESCRIPTION OF THE INVENTION

The following description of the disclosure is merely intended to illustrate various aspects of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent aspects are to be included herein. All references cited herein, including publications, patents and patent applications are incorporated herein by reference in their entirety.

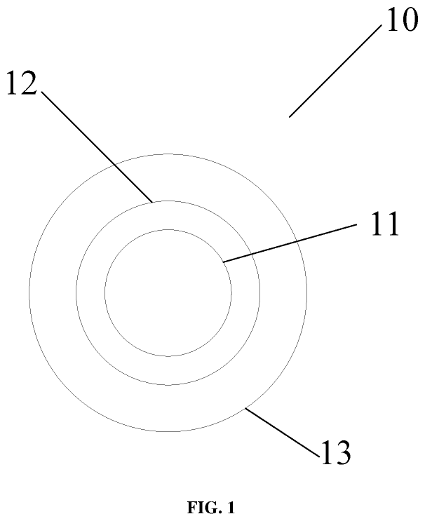

FIG. 1 illustrates a dosage form 10 according to an aspect of the present invention. The dosage form 10 includes a core geometric entity 11, a drug geometric entity 12 and a taste-masking geometric entity 13.

In some aspects, the dosage form 10 has a diameter or maximum length of about 334 μm to 500 μm. In other aspects, the dosage form 10 has a diameter or maximum length of about 417 μm. Dosage forms of this size have a more pleasant mouth feel for a patient for oral consumption.

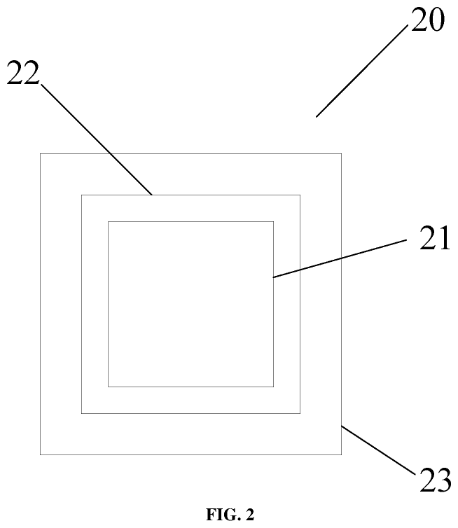

The dosage form 10 illustrated in FIG. 1 has a sphere shape. However, the dosage form 10 may have another shape, for example, a tablet shape, a capsule shape, a rod shape, a cubic or cuboidal shape, a pie shape, a cone shape, a pyramid shape, a cylindrical shape, a triangular or polygonal prism shape, and a tetrahedron shape, and the present invention is not limited to any of them. For example, FIG. 2 shows a dosage form 20 according to another embodiment, which includes a core geometric entity 21, a drug geometric entity 22 and a taste-masking geometric entity 23. Different from the dosage form 10 illustrated in FIG. 1, the dosage form 20 illustrated in FIG. 2 has a cubic shape.

In some aspects where the dosage form has a sphere shape, the dosage form may have a volume of about 2×10⁷ μm³ to 6.5×10⁷ μm³, and in some aspects, the dosage form has a volume of about 4×10⁷ μm³. In some aspects where the dosage form has a cubic shape, the dosage form may have a volume of about 3.7×10⁷ μm³ to 1.25×10⁸ μm³, and in a preferable embodiment, the dosage form has a volume of about 7×10⁷ μm³.

Referring back to FIG. 1, the core geometric entity 11 is used for providing a surface to apply the drug substance of the dosage form 10. The core geometric entity 11 may include, for example but not limited to, microcrystalline cellulose pellets, sugar spheres, starch spheres or other inert spherical pharmaceutically acceptable materials.

In some aspects, the core geometric entity 11 may have a diameter or maximum length of about 166 μm to 248 μm. In other aspects, the core geometric entity 11 may have a diameter or maximum length of about 207 μm. The core should be sized to allow a large amount of drug, while keeping the overall size of the dosage forms to a reasonable size.

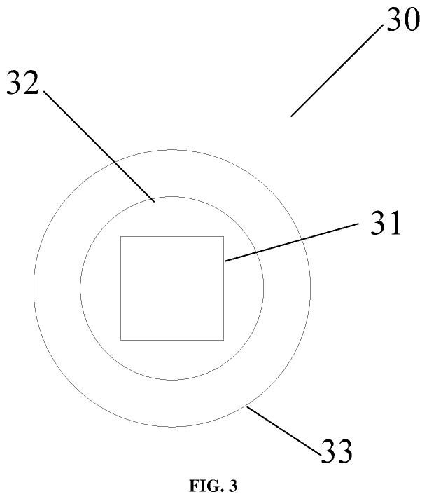

FIG. 1 shows that the core geometric entity 11 has a sphere shape. However, the core geometric entity 11 may have another shape, for example, a tablet shape, a capsule shape, a rod shape, a cubic or cuboidal shape, a pie shape, a cone shape, a pyramid shape, a cylindrical shape, a triangular or polygonal prism shape, and a tetrahedron shape, and the present invention is not limited to any of them. For example, FIG. 3 shows a dosage form 30 according to another aspect, which includes a core geometric entity 31, a drug geometric entity 32 and a taste-masking geometric entity 33. Different from the dosage form 10 illustrated in FIG. 1, the core geometric entity 31 included in the dosage form 30 illustrated in FIG. 3 has a cubic shape.

In some aspects where the core geometric entity has a sphere shape, the core geometric entity may have a volume of about 2.4×10⁶ m³ to 8×10⁶ m³, and in other aspects, the core geometric entity has a volume of about 4.6×10⁶ m³. In some aspects where the core geometric entity has a cubic shape, the core geometric entity may have a volume of about 4.6×10⁶ μm³ to 1.5×10⁷ μm³, and in other aspects, the dosage form has a volume of about 9×10⁶ μm³.

Referring back to FIG. 1, the drug geometric entity 12 comprises a drug substance. For example, the drug geometric entity 12 may comprise the sodium phenylbutyrate or any other drug substances that have unpleasant tastes.

In some aspects, the drug geometric entity 12 has an average thickness of about 77 μm to 115 μm. In other aspects, the drug geometric entity 12 has an average thickness of about 96 μm. A larger thickness for the drug geometric entity allows for more drug to be present in the dosage form. Higher drug loading is important when a large amount of drug substance is ingested.

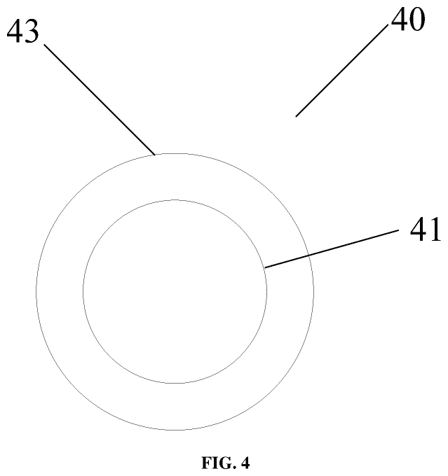

The drug geometric entity of the dosage form is connected with the core geometric entity. FIG. 1 shows that the drug geometric entity 12 is connected with the core geometric entity 11 by surrounding the core geometric entity 11. For example, the drug substance included in the drug geometric entity 12 may be prepared as a solution and then applied to the core geometric entity 11 by spraying followed by drying. However, in some aspects, the drug geometric entity of the dosage form may be connected with the core geometric entity by integrating with the core geometric entity. For example, FIG. 4 shows a dosage form 40, in which the drug geometric entity integrates with the core geometric entity as a drug-containing core 41. That is, the drug geometric entity is connected with the core geometric entity by integrating with the core geometric entity. In some aspects, the drug geometric entity may be connected with the core geometric entity by stacking on top of the core geometric entity or juxtaposing side by side with the core geometric entity.

Referring back to FIG. 1, the taste-masking geometric entity 13 is for inhibiting the release of the drug substance included in the drug geometric entity 12 from the dosage form 10 when the dosage form is administered orally into the subject, while allowing the release of the drug substance from the dosage form 10 in the stomach of the subject, so as to mask the unpleasant taste of the drug substance. In some aspects, the taste-masking geometric entity 13 includes compound(s) that results in a formulation that scores favorably in a taste test. Examples of such compounds include, but are not limited to, a copolymer of dimethylaminoethyl methacrylate, butyl methacrylate, and methyl methacrylate (e.g., Eudragit E PO). Other compounds that dissolve at the pH of the stomach but are insoluble at the pH of the mouth can also be used for preparing the taste-masking geometric entity 13.

In some aspects, the taste-masking geometric entity 13 has an average thickness of about 7 μm to 11 μm. In some aspects, the taste-masking geometric entity 13 has an average thickness of about 9 μm. A thinner layer keeps the overall size down and reduces the overall mass of the dosage form.

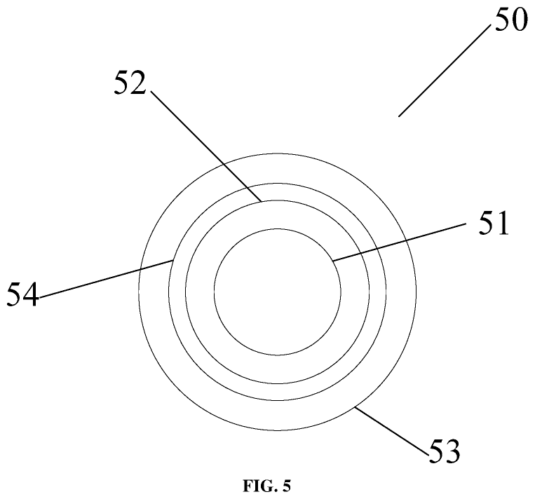

FIG. 5 illustrates a dosage form 50 according to another embodiment of the present invention. The dosage form 50 includes a core geometric entity 51, a drug geometric entity 52 and a taste-masking geometric entity 53, which have the same structures as those in the dosage form 10 and will not be repeatedly described herein.

Different from the dosage form 10 illustrated in FIG. 1, the dosage form 50 further includes a sealing geometric entity 54 applied between the drug geometric entity 52 and the taste-masking geometric entity 53, surrounding the drug geometric entity 52. The sealing geometric entity 54 is for preventing direct contact of the drug geometric entity 52 and the taste-masking geometric entity 53, so as to protect the drug substance included in the drug geometric 52 and the ingredients included in the taste-masking geometric entity 53 from deterioration. Examples of compounds which may be used in the sealing geometric entity 54 include, but are not limited to, Opadrys such as Opadry Clear, polyvinyl alcohols, hydroxyl-propylcellulose, hydroxypropylmethylcellulose, or polyvinylpyrrolidone.

In some aspects, the sealing geometric entity 54 has an average thickness of about 1 μm. A thinner layer keeps the overall size down and reduces the overall mass of the dosage form.

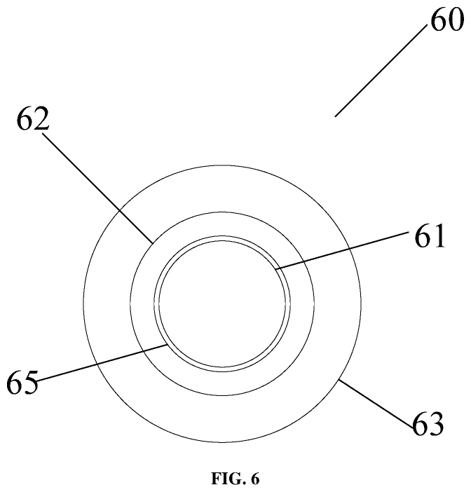

FIG. 6 illustrates a dosage form 60 according to an embodiment of the present invention. The dosage form 60 includes a core geometric entity 61, a drug geometric entity 62 and a taste-masking geometric entity 63, which have the same structures as those in the dosage form 10 and will not be repeatedly described.

Different from the dosage form 10 illustrated in FIG. 1, the dosage form 60 further includes a sealing geometric entity 65 applied between the drug geometric entity 62 and the core geometric entity 61. The sealing geometric entity 64 is for preventing direct contact of the drug geometric entity 62 and the core geometric entity 61, so as to protect the drug substance included in the drug geometric 62 and the ingredients included in the core geometric entity 61 from deterioration. Similar as the sealing geometric entity 54 illustrated in FIG. 5, examples of compounds which may be used in the sealing geometric entity 65 include, but are not limited to, Opadrys such as Opadry Clear, polyvinyl alcohols, hydroxyl-propylcellulose, hydroxypropylmethylcellulose, or polyvinylpyrrolidone.

In some aspects, the sealing geometric entity 65 has an average thickness of about 1 μm.

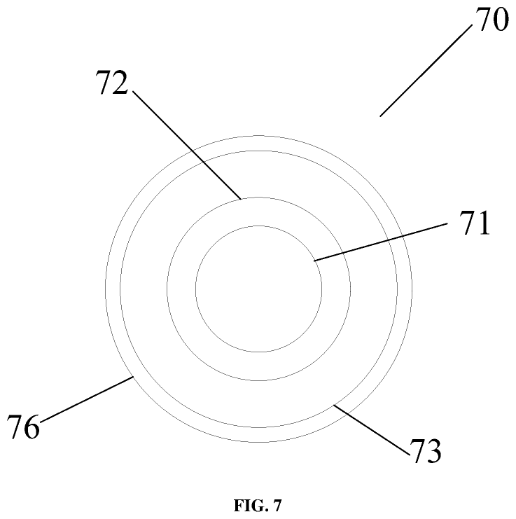

FIG. 7 illustrates a dosage form 70 according to an embodiment of the present invention. The dosage form 70 includes a core geometric entity 71, a drug geometric entity 72 and a taste-masking geometric entity 73, which have the same structures as those in the dosage form 10 and will not be repeatedly described herein.

Different from the dosage form 10 illustrated in FIG. 1, the dosage form 70 further includes a coating geometric entity 76 surrounding the taste-masking geometric entity 73. The coating geometric entity 76 is for preventing or reducing deterioration of the dosage form 70 by moisture in the air. Similar as the sealing geometric entity 54 illustrated in FIG. 5, examples of compounds which may be used in the coating geometric entity 76 include, but are not limited to, Opadrys such as Opadry Clear, polyvinyl alcohols, hydroxypropylcellulose, hydroxypropylmethylcellulose, or polyvinylpyrrolidone.

FIG. 5 to FIG. 7 illustrate multiple dosage forms in accordance with the present invention, which optionally include the sealing geometric entity 54 applied between the drug geometric entity and the taste-masking geometric entity, the sealing geometric entity 65 entity applied between the drug geometric entity and the core geometric entity, or the coating geometric entity 76 surrounding the taste-masking geometric entity. It can be understood by those skilled in the art that a dosage form according to the present invention can optionally include one, two or all of the sealing geometric entity 54, the sealing geometric entity 65 and the coating geometric entity 76 based on practical needs.

In some aspects, for a plurality of dosage forms that are described with respect to FIG. 1 to FIG. 7, 90% of the plurality of dosage forms have a volume of 7×10⁷ μm³ or lower and/or have a diameter or maximum length of 417 μm or lower. In some aspects, for a plurality of dosage forms that described with respect to FIG. 1 to FIG. 7, the core geometric entities included in 90% of the plurality of dosage forms have a volume of 9×10⁶ m³ or lower. In some aspects, for a plurality of dosage forms that described with respect to FIG. 1 to FIG. 7, the core geometric entities included in 90% of the plurality of dosage forms may have a diameter or maximum length of 207 μm or lower. In some aspects, for a plurality of dosage forms that described with respect to FIG. 1 to FIG. 7, the drug geometric entities included in 90% of the plurality of dosage forms may have a thickness of 96 μm or lower. In some aspects, for a plurality of dosage forms that described with respect to FIG. 1 to FIG. 7, the taste-masking geometric entities included in 90% of the plurality of dosage forms have a thickness of 9 μm or lower.

It is appreciated that the Summary and Abstract sections may set forth one or more, but not all exemplary aspects of the present invention as contemplated by the inventor(s), and thus, are not intended to limit the present invention and the appended claims in any way.

1. A dosage form comprising: a core geometric entity, a drug geometric entity connected with the core geometric entity, and a taste-masking geometric entity surrounding the drug geometric entity, wherein the drug geometric entity comprises a drug substance, and wherein the taste-masking geometric entity inhibits the rapid release of the drug substance from the dosage form when the dosage form is administered orally to the subject.

2. The dosage form according to claim 1, which has a shape selected from the group consisting of a sphere shape, a tablet shape, a capsule shape, a rod shape, a cubic or cuboidal shape, a pie shape, a cone shape, a pyramid shape, a cylindrical shape, a triangular or polygonal prism shape, and a tetrahedron shape.

3. The dosage form according to claim 1, which has a volume of 3.7×10⁷ μm³ to 1.25×10⁸ μm³.

4. (canceled)

5. The dosage form according to claim 1, which has a maximum length of 334 μm to 500 μm.

6. (canceled)

7. The dosage form according to claim 1, wherein the core geometric entity has a shape selected from the group consisting of a sphere shape, a tablet shape, a capsule shape, a rod shape, a cubic or cuboidal shape, a pie shape, a cone shape, a pyramid shape, a cylindrical shape, a triangular or polygonal prism shape, and a tetrahedron shape.

8. The dosage form according to claim 1, wherein the core geometric entity has a volume of 4.6×10⁶ μm³ to 1.5×10⁷ μm³.

9. (canceled)

10. The dosage form according to claim 1, wherein the core geometric entity has a maximum length of 166 μm to 248 μm.

11. (canceled)

12. The dosage form according to claim 1, wherein the drug geometric entity surrounds the core geometric entity.

13. The dosage form according to claim 12, wherein the drug geometric entity has a thickness of 77 μm to 115 μm.

14. (canceled)

15. The dosage form according to claim 1, wherein the taste-masking geometric entity has a thickness of 7 μm to 11 μm.

16. (canceled)

17. The dosage form according to claim 1, further comprising a first sealing geometric entity applied between the drug geometric entity and the taste-masking geometric entity, wherein the first sealing geometric entity prevents or reduces direct contact of the drug geometric entity and the taste-masking geometric entity.

18. The dosage form according to claim 17, wherein the first sealing geometric entity has a thickness of 1 μm.

19. The dosage form according to claim 1, further comprising a second sealing geometric entity applied between the core geometric entity and the drug geometric entity, wherein the second sealing geometric entity prevents or reduces direct contact of the core geometric entity and the drug geometric entity.

20. The dosage form according to claim 19, wherein the second sealing geometric entity has a thickness of 1 μm.

21. The dosage form according to claim 1, further comprising a powder coating geometric entity surrounding the taste-masking geometric entity, wherein the powder coating geometric entity prevents or reduces deterioration of the dosage form by moisture in the air.

22. A dosage form comprising: a core geometric entity, a drug geometric entity surrounding the core geometric entity, a first sealing geometric entity surrounding the drug geometric entity, and a taste-masking geometric entity surrounding the first sealing geometric entity, wherein the drug geometric entity comprises a drug substance, wherein the first sealing geometric entity prevents direct contact of the drug geometric entity and the taste-masking geometric entity, and wherein the taste-masking geometric entity inhibits the rapid release of the drug substance from the dosage form when the dosage form is administered orally to the subject.

23. The dosage form of claim 22, which has a shape selected from the group consisting of a sphere shape, a tablet shape, a capsule shape, a rod shape, a cubic or cuboidal shape, a pie shape, a cone shape, a pyramid shape, a cylindrical shape, a triangular or polygonal prism shape, and a tetrahedron shape.

24. The dosage form according to claim 22, which has a volume of 3.7×10⁷ μm³ to 1.25×10⁸ μm³.

25. (canceled)

26. The dosage form according to claim 22, which has a maximum length of 334 μm to 500 μm.

27. (canceled)

28. The dosage form according to claim 22, wherein the core geometric entity has a shape selected from the group consisting of a sphere shape, a tablet shape, a capsule shape, a rod shape, a cubic or cuboidal shape, a pie shape, a cone shape, a pyramid shape, a cylindrical shape, a triangular or polygonal prism shape, and a tetrahedron shape.

29. The dosage form according to claim 22, wherein the core geometric entity has a volume of 4.6×10⁶ μm³ to 1.5×10⁷ μm³.

30. (canceled)

31. The dosage form according to claim 22, wherein the core geometric entity has a maximum length of 166 μm to 248 μm.

32. The dosage form according to claim 31, wherein the core geometric entity has a maximum length of 207 μm.

33. The dosage form according to claim 22, wherein the drug geometric entity has a thickness of 77 μm to 115 μm.

34. (canceled)

35. The dosage form according to claim 22, wherein the first sealing geometric entity has a thickness of 1 μm.

36. The dosage form according to claim 22, wherein the taste-masking geometric entity has a thickness of 7 μm to 11 μm.

37. (canceled)

38. The dosage form according to claim 22, further comprising a second sealing geometric entity applied between the core geometric entity and the drug geometric entity, wherein the second sealing geometric entity prevents or reduces direct contact of the core geometric entity and the drug geometric entity.

39. The dosage form according to claim 38, wherein the second sealing geometric entity has a thickness of 1 μm.

40. The dosage form according to claim 22, further comprising a powder coating geometric entity surrounding the taste-masking geometric entity, wherein the powder coating geometric entity prevents or reduces the dosage form from deterioration by moisture in the air.

41. A plurality of dosage forms, each dosage form comprising: a core geometric entity, a drug geometric entity connected with the core geometric entity, and a taste-masking geometric entity surrounding the drug geometric entity, wherein the drug geometric entity comprises a drug substance, and wherein the taste-masking geometric entity inhibits the rapid release of the drug substance from the dosage forms when the dosage forms are administered orally to the subject.

42. A plurality of dosage forms according to claim 41, which have shapes selected from the group consisting of a sphere shape, a tablet shape, a capsule shape, a rod shape, a cubic or cuboidal shape, a pie shape, a cone shape, a pyramid shape, a cylindrical shape, a triangular or polygonal prism shape, and a tetrahedron shape.

43. A plurality of dosage forms according to claim 41, wherein 90% of the plurality of dosage forms have a volume of 7×10⁷ μm³ or lower.

44. A plurality of dosage forms according to claim 41, wherein 90% of the plurality of dosage forms have a maximum length of 417 μm or lower.

45. A plurality of dosage forms according to claim 41, wherein the core geometric entities have a shape selected from the group consisting of a sphere shape, a tablet shape, a capsule shape, a rod shape, a cubic or cuboidal shape, a pie shape, a cone shape, a pyramid shape, a cylindrical shape, a triangular or polygonal prism shape, and a tetrahedron shape.

46. A plurality of dosage forms according to claim 41, wherein the core geometric entities included in 90% of the plurality of dosage forms have a volume of 9×10⁶ μm³ or lower.

47. A plurality of dosage forms according to claim 41, wherein the core geometric entities included in 90% of the plurality of dosage forms have a maximum length of 207 μm or lower.

48. A plurality of dosage forms according to claim 41, wherein for each dosage form, the drug geometric entity surrounds the core geometric entity.

49. A plurality of dosage forms according to claim 48, wherein the drug geometric entities included in 90% of the plurality of dosage forms have a thickness of 96 μm or lower.

50. A plurality of dosage forms according to claim 41, wherein the taste-masking geometric entities included in 90% of the plurality of dosage forms have a thickness of 9 μm or lower.

51. A plurality of dosage forms according to claim 41, each dosage form further comprising a first sealing geometric entity applied between the drug geometric entity and the taste-masking geometric entity, wherein the first sealing geometric entity prevents or reduces direct contact of the drug geometric entity and the taste-masking geometric entity.

52. A plurality of dosage forms according to claim 51, wherein the first sealing geometric entities have a thickness of 1 μm or lower.

53. A plurality of dosage forms according to claim 41, each dosage form further comprising a second sealing geometric entity applied between the core geometric entity and the drug geometric entity, wherein the second sealing geometric entity prevents or reduces direct contact of the core geometric entity and the drug geometric entity.

54. A plurality of dosage forms according to claim 53, wherein the second sealing geometric entities have a thickness of 1 μm or lower.

55. A plurality of dosage forms according to claim 41, each dosage form further comprising a powder coating geometric entity surrounding the taste-masking geometric entity, wherein the powder coating geometric entities prevent or reduce deterioration of the dosage forms by moisture in the air.
